(19) United States
(12) Patent Application Publication
(10) Pub. No.: US 2006/0141486 A1
(43) Pub. Date: Jun. 29, 2006

(54) Porous substrates and arrays comprising the same

(76) Inventors: Everett W. Coonan, Painted Post, NY (US); Ye Fang, Painted Post, NY (US); Ann M. Ferrie, Painted Post, NY (US); Xiaodong Fu, Painted Post, NY (US); Yulong Hong, Painted Post, NY (US); Thomas M. Leslie, Horseheads, NY (US); Xinghua Li, Horseheads, NY (US); Beth C. Monahan, Painted Post, NY (US); Eric J. Mozdy, Elmira, NY (US); Dirk Muller, Lafayette, CO (US); Cameron W. Tanner, Horseheads, NY (US); Patrick D. Tepesch, Corning, NY (US); John F. Wight JR., Corning, NY (US); Po Ki Yuen, Corning, NY (US)

Correspondence Address: CORNING INCORPORATED, SP-TI-3-1, CORNING, NY, 14831, US

(21) Appl. No.: 11/027,400

(22) Filed: Dec. 29, 2004

(51) Int. Cl. C12Q 1/68; G01N 33/53; C12M 1/34; B05D 3/02

(52) U.S. Cl. 435/6; 435/7.1; 435/287.2; 427/2.11

(57) ABSTRACT

The present invention relates to arrays comprising porous substrates for attachment of nucleic acids, polypeptides, membranes, or other biological or organic materials. In many embodiments, the arrays of the present invention have a flow-through configuration such that washing buffers or samples can access to the porous substrates from at least two sides of the arrays. The present invention also features arrays comprising UV-compatible porous substrates, arrays comprising three-dimensional membranes in sol-gels, and arrays comprising silica-based porous substrates prepared using a low-temperature fusion process.

### FIELD OF THE INVENTION

The present invention relates to arrays comprising porous substrates for attachment of nucleic acid, polypeptides, membranes or other biological or organic materials.

### BACKGROUND OF THE INVENTION

Microarrays allow for quantitative detection of a large number of genes or proteins at one time. Traditional microarrays are performed on planar, non-porous surfaces (i.e., 2-D surfaces) upon which probes are either deposited directly or synthesized in situ. The use of 2-D surface has numerous limitations. For example, hybridization on a 2-D surface is often time-consuming; the probe accessibility and loading capacity are relatively low; and the area available for hybridization or reaction is limited. In addition, the inherent geometric constraint of the 2-D surface makes traditional microarrays an unappealing platform for the analysis of membrane proteins, such as G-protein coupled receptors (GPCRs), ion channels, or other membrane-bound drug targets.

PCT Applications W00116376 and W00061282 describe the use of porous materials as substrates for making DNA microarrays. Porous substrates offer several advantages compared to two-dimensional substrates. For example, porous substrates can achieve improved probe loading capacity, enhanced target binding specificity, greater accessibility of targets to the probes, and reduced reaction/hybridization time. Furthermore, porous substrates provide a superior platform for the analysis of membrane proteins, allowing simultaneous detection of ligand binding at one side of a membrane and activation/inactivation of downstream effector(s) on the other side. However, several drawbacks have been demonstrated for this type of substrates. For example, washing of porous substrates after reaction is frequently inefficient; and automation of the washing and drying steps has been difficult to implement. Therefore, there is a need to make new arrays that would overcome these shortcomings.

### SUMMARY OF THE INVENTION

The present invention provides arrays comprising porous substrates for attachment of nucleic acids, polypeptides, membranes, or other biological or organic materials. In many embodiments, the arrays of the present invention have a flow-through configuration, allowing washing buffers or samples to access to the porous substrates from at least two sides of the arrays. This configuration significantly improves the washability of the porous substrates and facilitates automation of the array analysis. The present invention also features arrays comprising UV-compatible substrates, arrays comprising three-dimensional membranes in sol-gels, and arrays comprising silica-based porous substrates prepared at low temperatures.

In one aspect, the present invention provides arrays comprising at least one substrate support and a plurality of discrete regions, each discrete region comprising a porous substrate attached to or supported by the substrate support(s). Each of these arrays has a flow-through configuration such that samples or wash buffers can assess to the porous substrate from at least two sides of the array (e.g., from two opposite sides of the array, such as a top side and a bottom side).

In one embodiment, the porous substrate is attached to or supported by a surface of a substrate support. The substrate support comprises one or more channels which pass through the substrate support from the porous substrate-associated surface to a surface opposite thereto. Samples or wash buffers can communicate from this opposite surface to the porous substrate through the channel(s). In many cases, communication through the channel(s) is operated in a controllable manner such that sample or fluid conveyance through the channel(s) occurs only during desired step(s) (e.g., washing step). For example, communication through the channels can be restricted such that samples or solutions are retained on one side of the array. Samples or solutions can also be driven through the channel(s) by using an external physical force (such as, by air-pressure or vacuum).

In another embodiment, an array of the present invention comprises a microplate including a plurality of wells, each well comprising a porous substrate. For each well, the microplate comprises one or more channels that connect the well to the bottom surface of the microplate. Samples or wash buffers can communicate from the porous substrate-attachment side to another surface of the support substrate through these channels.

In still another embodiment, an array of the present invention comprises a holey microplate including a plurality of openings. A porous substrate is positioned in each of these openings such that samples or wash buffers can access to both sides of the porous substrate.

In a further embodiment, an array of the present invention comprises two holey plates, between which a porous material sheet is sandwiched. The holes of these two plates are aligned to expose discrete regions on the porous material sheet such that samples or wash buffers can access to these discrete regions from both sides of the array.

Any organic, inorganic or biological material may be attached to or associated with the porous substrates of the present invention. For instance, nucleic acids, polypeptides, polysaccharides, lipids, cells, cell components, tissues, or tissue parts can be stably associated with a porous substrate of the present invention. In one embodiment, a porous substrate comprises or is stably associated with a membrane, such as a biological membrane or an artificially reconstituted membrane. In many cases, the membrane comprises one or more membrane proteins, such as G protein coupled receptors (GPCRs), ion channels, transporters, or kinase receptors. Structural or functional analyses of these membrane proteins can be performed using an array of the present invention.

Any porous material may be used to make the porous substrates of the present invention. In many embodiments, the porous substrates comprise or consist essentially of anodic aluminum oxide, fused silica or sol-gel.

In one aspect, the porous substrates employed in the present invention are gelation products of mixtures that comprise sol-gel precursors and membranes. Suitable sol-gel precursors for this purpose include, but are not limited to, tetraalkoxysilanes or trialkoxysilanes. In one embodiment, an array of the present invention is fabricated according to the following steps:

mixing at least one sol-gel precursor with a membrane;

hydrolyzing the sol-gel precursor(s) to form a sol-gel including the membrane; and

depositing the sol-gel into discrete regions on a substrate support.

In another embodiment, an array of the present invention is fabricated according to the following steps:

mixing at least one sol-gel precursor with a membrane under conditions that no significant gelation occurs;

depositing the mixture of the sol-gel precursor and membrane into discrete regions on a substrate support; and

initiating gelation in the discrete regions to form sol-gels including the membrane.

In another aspect, the porous substrates employed in the present invention are fusion products of mixtures that comprise silica beads and silanes. Suitable silanes for this purpose include, but are not limited to, 3-acyloxypropyl-trimethoxysilane, allyltrichlorosilane, 3-aminpropyltriethoxysilane, N-(6-aminohexyl)aminopropyl-trimethoxysilane, bis(triethoxysilye)methane, 2-(3-cyclohexenyl)ethyl)triethoxysilane, 3-glycidoxypropyl-trimethoxysilane, and tetramethoxysilane.

In one embodiment, an array of the present invention is prepared according to the following steps:

formulating silica beads in an organic solvent comprising at least one silane;

depositing the formulated silica beads into discrete regions on a substrate support; and

curing the substrate support to fuse the silica beads to form porous substrates in the discrete regions.

In many cases, the curing process is performed at a temperature of no greater than about 200° C., such as at room temperature. The concentration of silane(s) in a formulated silica bead mixture can be, without limitation, from about 0.01% to about 10% by volume. Because of the low-temperature fusion process, polymeric, inorganic, metal or other materials may be used as substrate supports for attachment of the porous substrates. The silica beads can be of any shape, e.g., spherical or irregular.

In still another aspect, the porous substrates employed in the present invention are UV-compatible. Examples of UV-compatible materials include, but are not limited to, silica-based glass, fused silica, calcium fluoride, or sapphire. In one embodiment, the UV-compatible porous substrates consist essentially of substantially pure fused silica. The particle size of the substantially pure fused silica may range, for example, from about 1 nm to about 5 μm, or preferably, from about 0.3 μm to about 1.5 μm. In one example, the substantially pure fused silica consists essentially of silica beads with particle sizes of about 1.0 μm. The substrate supports employed in the present invention can also be UV-compatible. The substrate supports can be made from the same materials that are used for making the UV-compatible porous substrates.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The drawings are provided for illustration, not limitation.

FIGS. 1A, 1B and 1C schematically illustrate two examples of nano-porous microplates. FIG. 1A depicts a microplate format, and FIGS. 1B and 1C illustrate two different forms of porous anodic aluminum oxide.

FIGS. 2A and 2B schematically illustrate a stand-alone porous microplate. FIG. 2A shows a microplate format, and FIG. 2B demonstrates the configuration of a well of the microplate.

FIGS. 3A and 3B schematically depict a microchanneled porous microplate. FIG. 3A shows a microplate format, and FIG. 3B depicts the configuration of a well of the microplate.

FIGS. 4A and 4B indicate the superior performance of a flow-through microplate for G protein-coupled receptor (GPCR) arrays on γ-aminopropylsilane (GAPS) coated porous substrate. Interactions between human muscarinic receptor subtype 1 (M1), human delta opioid receptor subtype 2 (delta2) or human muscarinic receptor subtype 2 (M2) and a mixture of labeled ligands containing 2 nM Cy3B-telenzepine and 4 nM Cy5-naltrexone were evaluated in the absence (FIG. 4A) or presence of (FIG. 4B) unlabeled telenzepine (2 μM) and naltrexone (4 μM).

FIGS. 5A and 5B further illustrate the superior performance of a flow-through microplate for GPCR arrays on GAPS porous substrate. FIG. 5A indicates the average fluorescence intensities of M1, delta2 or M2 receptors in the array assays described in FIGS. 4A and 4B. FIG. 5B shows the fluorescence intensities of delta2 receptor as a function of microspots. RFU: relative fluorescence unit.

FIGS. 6A-6E illustrate fluorescence signals of UV-excited europium chelates mixed with silica powders of different particle sizes. FIG. 6A shows fluorescence signal using a non-pure silica power, and FIGS. 6B-6E show fluorescence signals using silica powders with the particle size of 0.3 μm, 0.5 μm, 1.0 μm or 1.5 μm, respectively. A 13-fold enhancement in fluorescence signal was detected without a significant increase in background signal (compare 5,000 to 65,000 signal count). The optimum particle size for europium-chelate fluorescence is around 1.0-μm diameter (FIG. 6D). The “coffee-ring” like structure stems from the drying process of the deposited solution.

FIGS. 7A and 7B show time-resolved fluorescence from eu-GTP dye printed on two different porous substrates. FIG. 7A used a traditional glass composition that has not been optimized for UV transmission, while FIG. 7B used pure fused silica beads. Both porous surfaces were fabricated by screen printing a slurry of micron-sized particles onto a substrate of similar composition, then sintering the sample to lock the particles to the surface. Both samples were printed at the same time, using the same size quill pin, pulling sample from the same container. These figures show the benefit of the UV-compatible material for lower background fluorescence. In this test, the three spots printed on the traditional surface displayed a signal-to-background of about 1.08, while those on the fused silica surface show a signal-to-background of about 1.56.

### DETAILED DESCRIPTION OF THE INVENTION

The present invention relates to arrays comprising porous substrates for attachment of nucleic acids, polypeptides, membranes, or other biological or organic materials. In many embodiments, the arrays of the present invention have a flow-through configuration such that washing buffers or samples can access to the porous substrates from at least two sides of the arrays. The present invention also features arrays comprising UV-compatible porous substrates, arrays comprising three-dimensional membranes in sol-gels, and arrays comprising silica-based porous substrates prepared using a low-temperature fusion process.

It is to be understood that the present invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a” and “an” include plural reference unless the context clearly dictates otherwise, and the use of “or” means “and/or” unless stated otherwise. The use of subsections is not meant to limit the invention; each subsection may apply to any aspect of the invention.

I. Porous Substrates

In one embodiment, an array of the present invention comprises a porous aluminum oxide layer. Aluminum oxide nano-porous substrates, such as anodic aluminum oxide, have been used to make microchannel plates (MCP). MCP is a matrix of parallel microchannels which cross one side of the plate to the other without interchannel connection. Self-organized anodic aluminum oxide can be formed by electrochemical oxidation of aluminum or aluminum alloy in electrolytes that weakly dissolve aluminum. The aluminum oxide thus-produced consists of regular hexagonally packed cells, which are parallel to each other and perpendicular to the surface of the aluminum substrate. See, for example, Delendik and Voitik, “Anodic Alumina as Material for High-Aspect Ratio Microstructures,” PROCEEDING OF FOURTH INTERNATIONAL WORKSHOP ON HIGH-ASPECT-RATIO MICRO-STRUCTURE TECHNOLOGY, June 2001 (Baden-Baden, Germany, 2 pp.); and Govyadinov, et al., “Anodic Aluminum Oxide Microchannel Plates,” Nuclear Instruments and Methods in Physics Research, A 419: 667-675 (1998), both of which are incorporated herein by reference in their entireties.

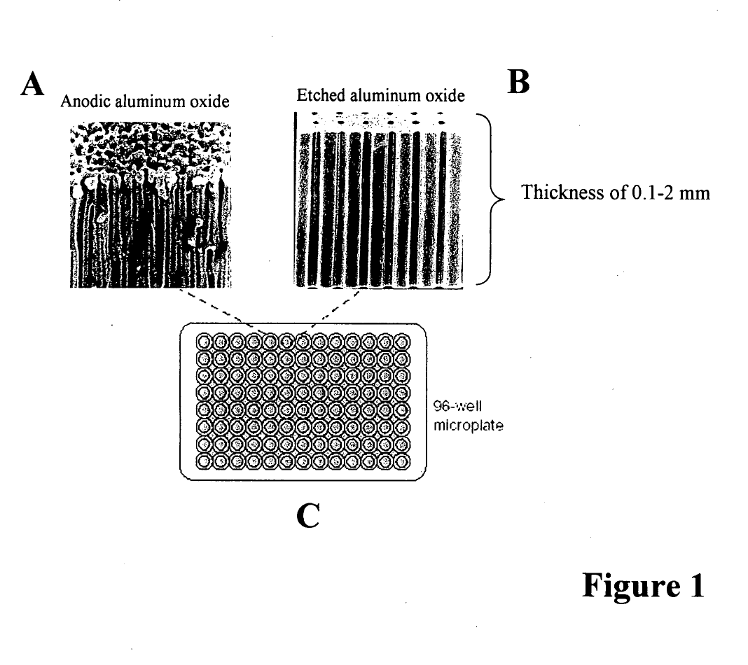

Each cell in an aluminum oxide porous sheet has an axial pore, closed by the barrier oxide layer on the side of aluminum anode (see, e.g., FIG. 1A). In many cases, the pore diameter is tunable by variation of the electrolyte composition or other anodization conditions. The pore diameter can be enlarged by selective etching of cell walls (see, e.g., FIG. 1B). The diameters of the microchannels thus-produced can range from a few nanometers (e.g., about 5, 10, or 20 nm) to several hundred nanometers (e.g., about 300, 400, or 500 nm), while the thickness of the porous aluminum oxide layer can be varied from less than 100 nm to over 500 micrometers (e.g., about 2 mm). Channels with greater diameters can also be produced by means of additional processing based on the intrinsic microchannel structures.

In one example, nano-porous anodic alumina layers are grown in a solution of an organic or inorganic acid. Suitable acids for this purpose include, but are not limited to, sulfuric acid, phosphoric acid, oxalic acid, chromic acid, boric acid, citric acid, or a mixture thereof. The concentration of the electrolyte can range, without limitation, from 0.1 to 99.9% by weight, or preferably, from 2 to 20% by weight. The temperature of the electrolyte can range, without limitation, from −90° C. to +150° C., or preferably, from −20° C. to +35° C. The anodization voltage can range, without limitation, from 0.5V to 500V, or preferably, from 5V to 100V.

Electrolyte, temperature and anodization voltage may be varied depending on the desired parameters of the anodic alumina substrate, such as thickness, pore diameter, pore density, surface area, type and concentration of impurities. The pore diameter, for example, is observed to depend on the anodization voltage. The pore density is observed to depend on the type of electrolyte used. The pore size is observed to decrease with decreasing the anodization voltage, while the layer growth rate is observed to depend on the desired pore diameter and electrolyte composition, and is proportional to the current density. The layer thickness is observed to be proportional to the charge density.

Aluminum foil or aluminum film on supporting substrates that preferably comprises at least 95% by weight aluminum, more preferably at least 99% by weight aluminum, and even more preferably at least 99.9% by weight aluminum, may be used for anodization. Prior to anodization, aluminum samples are preferably degreased and pressure annealed. Graphite, lead or aluminum plates may be used as counter electrodes. Anodization with constant voltage/current or with voltage/current modulated at high frequency may be used to produce pores of diameter uniform throughout the thickness of the film. More complex process profiles of anodization voltage, current and/or temperature may also be used for the preparation of nano-porous alumina films. Changing process parameters at a low frequency (10 Hz and lower) may be used to fabricate pores with modulated diameter and density.

A dense oxide barrier layer normally separates the bottom of the pores from the underlying aluminum substrate. There are a variety of techniques for reducing and removing these insulation layers from anodic alumina substrates including gradually reducing the cell voltage and then chemically dissolving the resulting thin barrier layer. The barrier layer in this case is pierced with small pores. This type of anodic alumina is referred to as “asymmetric” due to the different size of the pores at the top and bottom surfaces.

Another technique is to apply cathodic polarization to the aluminum substrate upon which the anodic alumina substrate is formed. Cathodic voltage or current may be less than, equal to or greater than the value of the anodization voltage and current. This cathodic polarization leads to rapid electrochemical dissolution of the barrier layer and separation of the anodic alumina substrate from the aluminum substrate. These films have the same pore diameter at both faces and are therefore referred to as “symmetric.” The electrolyte for this process may be the same as the anodization electrolyte or may be a different electrolyte preferably comprising strong acids. For example, perchloric, acetic, phosphoric acids, or mixtures thereof can be used. A combination of these techniques can also be used.

The present invention also contemplates inclusion of other desirable microstructures on an anodic alumina sheet, such as raised or depressed regions, trenches, v-grooves, mesa structures, or other regular or irregular configurations. Microfabrication on anodic alumina can be performed, for example, by anisotropic etching, localized anodization, or by combination thereof. In combination, these techniques enable versatile and flexible combination of bulk- and surface-like microstructures, creating powerful design and application opportunities.

The porous and compositional anisotropy of anodic alumina allows anisotropic etching of anodic alumina, with etchant species penetrating the entire thickness of the film and etching sideways. In one example, the processing sequence comprises: (1) anodizing aluminum to form nano-porous anodic alumina films of required thickness and morphology; (2) depositing a protective thin film to close the pores to prevent the penetration of the photoresist deep inside the pores, where this thin film preferably includes metals (such as aluminum, copper, nickel, molybdenum, tantalum, niobium, and their alloys), metal oxides, or other thin films; (3) applying and pre-baking a photoresist; (4) exposing and developing the photoresist; (5) hard-baking photoresist pattern; (6) etching protective film; (7) anisotropically etching anodic alumina substrate in exposed areas of the film in the liquid or gas-phase process (e.g., in the solution of phosphoric and chromic acids at temperature from 0° C. to 100° C., preferably from 50° C. to 95° C.); (8) striping photoresist and protective layers from resulting micromachined pattern; (9) separating the micromachined anodic alumina substrate from aluminum by selective dissolution of aluminum; and (10) rinsing and drying the resulting micromachined substrate.

In another approach, desirable microstructures on anodic alumina can be made by localized anodization, followed by selective etching of aluminum to release the resulting microstructures. This technique comprises the steps of: (1) pre-anodization of aluminum to form a thin layer (e.g., 100-250 nm) of anodic alumina to increase the adhesion of the photoresist; (2) application of the photolithographic mask as described above; (3) anodization to form nano-porous anodic alumina substrates of required thickness and morphology; (4) striping photoresist and protective layers from resulting pattern; and (5) separating anodic alumina substrates from aluminum substrate by selective dissolution of aluminum.

A porous anodic alumina sheet prepared according to the present invention can be annealed to increase its surface area and chemical, mechanical or thermal stability. Annealing can be performed in air, preferably at temperatures greater than 500° C., and more preferably in the range at 750° C. to 1200° C.

The surface(s) of anodic aluminum oxide or other types of porous substrates employed in the present invention can be modified to facilitate attachment or immobilization of organic or biological molecules. A surface of a porous substrate can include an external surface of the substrate, or an internal surface that is located in the pores of the substrate. A variety of methods can be used to deposit materials onto or inside anodic alumina or other porous substrates. These methods include, but are not limited to, spin coating, dip coating, spray coating, solution impregnation, physical sputtering, reactive sputtering, physical vapor deposition, chemical vapor deposition, atomic layer chemical vapor deposition via binary reaction sequences, ion beam, e-beam deposition, molecular beam epitaxy, laser deposition, plasma deposition, electrophoretic deposition, magnetophoretic deposition, thermophoretic deposition, stamping, centrifugal casting, gel casting, extrusion, electrochemical deposition, screen and stencil printing, brush painting, or a combination thereof.

The surface(s) of anodic alumina substrate or other porous substrates employed in the present invention can be coated with one or more modification layers. Suitable modification layers include inorganic or organic layers, such as metals, metal oxides, alloys, ceramics, polymers, bifunctional or cross-linking agents, small organic molecules, bio-organisms, biologically active materials, biologically derived materials, or combinations thereof. In many instances, the surface(s) of a porous substrate is chemically or physically treated to include groups such as hydroxyl, carboxyl, amine, aldehyde, or sulfhydryl moieties, or their modified forms. These derivatized functional groups allow stable attachment of nucleic acids, polypeptides, lipids or other biological molecules to the porous substrate. The modification layer(s) can be covalently or non-covalently attached to the surface(s) of a porous substrate.

Anodic aluminum oxide or other porous substrates are preferably attached to a substrate support. Substrate supports suitable for the present invention include, but are not limited to, glass, silica, ceramic, nylon, quartz wafer, metal, paper, gel, and other solid or semi-solid materials. The substrate supports can be flexible or rigid. In many embodiments, the substrate supports are non-reactive with reagents that are used in array assays. Any method know in the art may be used to attach a porous substrate to a substrate support. Substrate supports are frequently used to provide physical support in order to overcome the fragility of the porous substrates, and thereby protect the integrity of the porous substrates. In many embodiments, the substrate supports employed in the present invention contain at least one channel across the support. The channel is preferably vertically across the support with a small dimension. The diameter of the channel can be, without limitation, from 10 to 1000 microns; such as from 100 to 500 microns.

In one embodiment, an array of the present invention is prepared by sandwiching an anodic alumina sheet between two holey plates (such as a holy microplate and a polyethylene sheet). In many cases, these two holey plates have the same or similar multiple-hole format. Alignment of these two holey plates creates regions in which both sides of the anodic alumina sheet is accessible for samples or wash buffers. As appreciated by those of ordinary skill in the art, these regions can have any desired size, shape, density, or spatial arrangement.

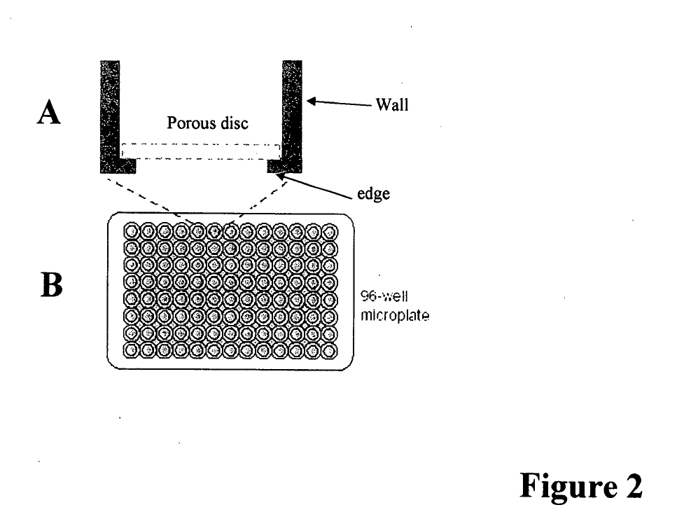

In addition to anodic alumina, fused silica or other porous substrates, such as those described in PCT publications WO0061282 and WO0116376, both of which are incorporated herein by reference in their entireties, can also be used to make the arrays of the present invention. In one embodiment, an array of the present invention comprises a holey microplate having a plurality of openings. A stand-alone porous substrate patch, such as a fused silicate or anodic alumina patch (see, e.g., FIGS. 2A and 2B), is positioned in each of these openings. The porous substrate patch may be of any shape or size, and can be positioned at any location in the opening. Samples or wash buffers can freely access to the porous substrate patch from both sides of the array.

A porous substrate can be held in an opening by any suitable means. In one example, the porous substrate patch is supported by the extended edge(s) at the bottom of an opening (see, e.g., FIG. 2A). The porous patch can be readily removable from the opening. The porous patch can also be stably affixed to the opening (such as through bonding to the surfaces or substructures in the opening).

Any sized or shaped opening may be employed in the present invention. The openings in a substrate support can be in any format, and the distance between each two openings may be in any desired range.

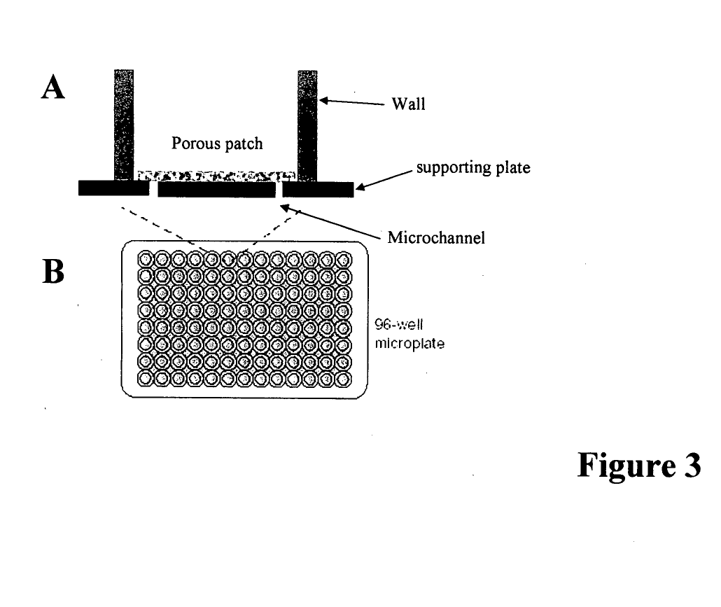

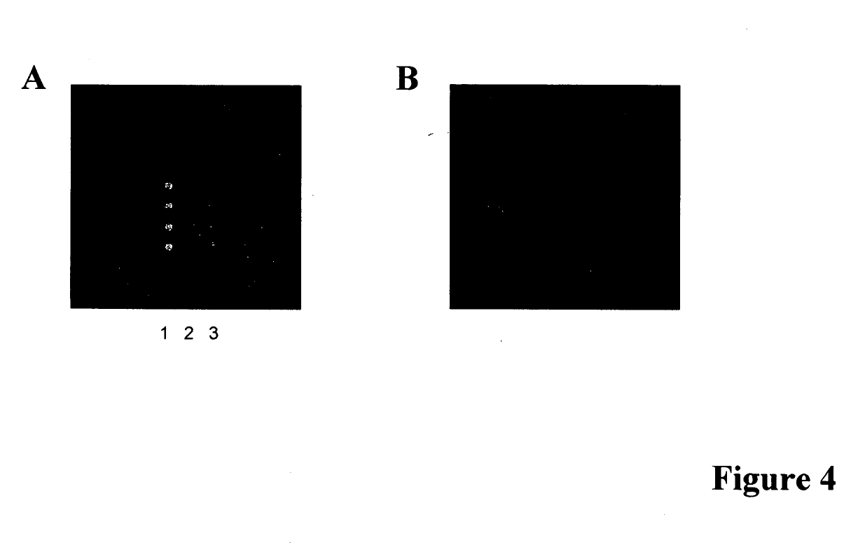

In another embodiment, an array of the present invention comprises a substrate support (e.g., a glass or polymer plate) coated or stably associated with a plurality of porous substrate islands. See, e.g., FIGS. 3A and 3B. The porous substrate islands are located in predetermined regions on the substrate support. Below each porous substrate patch, there is at least one channel which passes through the substrate support from the porous substrate-attached surface to a surface opposite thereto. See, e.g., FIGS. 4A and 4B. Samples or wash buffers can access from the opposite surface to the porous substrate patch through the channel.

Channels can be created in a substrate support by using any method known in the art, including but not limited to various etching or injection molding techniques. The choice of the methods to make the channel is dependent on the type and nature of the support substrate. For example, for polymeric or ceramic substrates, laser drilling or injection molding methods are preferred, whereas for glass or metal supports, sand blasting methods are preferred. The size of each channel may be in any range, such as from less than 50 μm to over several millimeters. In many cases, at least 2, 3, 4, 5 or more channels are constructed nearby or underneath a porous substrate to provide access to the substrate. The use of channels underlying porous substrates combines the advantages of porous materials and filter-based biological separation devices.

Each porous substrate patch employed in the present invention may have any desired size or shape. The porous substrate patches on a substrate support can be organized into any desired form or pattern.

In one example, a silica-based porous flow-through microplate is fabricated according to the following steps:

sand blasting or laser drilling to make a plurality of channels at predetermined locations on a 1737 glass plate (Corning Inc.);

screening printing to print patches of silicate frits to these predetermined locations;

high temperature sintering (e.g., at about 700° C.) to consolidate frits to form porous substrates; and

assembling the 1737 glass plate into a microplate.

In another example, a flow-through microplate is fabricated according to the following steps:

injection molding to make a plurality of channels at predetermined regions on a substrate support (e.g., a glass or polymer plate);

reformulating silicate frits with silanes;

screening printing to print patches of sol-gels containing the silicate frits and silanes to the predetermined regions on the substrate support;

sintering at low temperatures (e.g., from about 100° C. to about 200° C.) to consolidate the silicate frits to form porous substrates; and

automated assembling the substrate support to form a microplate.

In still another example, a stand-alone porous disc-based microplate is fabricated according to the following steps:

injection molding to make a holey microplate containing recess areas in predetermined regions of the side wall of each well;

screen printing to deposit patches of silicate frits to a metal support in the predetermined regions;

sintering at desired temperatures (e.g., from about 650° C. to about 750° C.; preferably from about 690° C. to about 715° C.) to consolidate the silicate frits to form standalone porous substrates; and

placing the standalone porous substrates into the recess area of the holey plate to form a microplate.

In this embodiment, because the porous substrates are not attached to the metal support during the sintering step, the porous substrates (e.g., porous discs) can be easily removed from the metal support after sintering and then transferred to the holey plate where the discs can fit in the recess region of the side wall of each well.

In a further example, a flow-through polymeric microplate comprising polymeric porous substrates is fabricated according to the following steps:

injection molding to make channels at predetermined locations on a polymeric substrate support;

placing or attaching polymeric porous substrates to the predetermined locations; and

assembling the polymeric substrate support and the polymeric porous substrates to form a microplate by either thermal bonding or adhesive chemistry.

In yet another example, an all glass-based flow-through microplate is prepared according to the following steps:

conducting positional etching of a glass substrate to form separate porous substrate patches at predetermined locations such that only the top layer(s) of the glass substrate becomes porous;

sand blasting or laser drilling to prepare at least one vertically channel underneath each porous substrate patch at the predetermined locations such that the channel passes through the substrate; and

automated assembling the substrate having separate porous patches and corresponding underneath channels to form a microplate.

In many embodiments, the porous substrates employed in the present invention are prepared from silica, fused silica or anodic alumina. Numerous methods are available for attaching porous materials to a substrate support (e.g., a glass or polymer plate). For instance, high temperature-induced fusion processes can be used to consolidate silica beads to form porous materials, followed by attaching the fused silica to predefined regions on a substrate support.

The present invention also features the use of silanes to reformulate the silica-bead suspension, followed by printing or depositing the mixture of silica-beads and silanes at predefined locations on a substrate support. The substrate support is then cured under conditions that allow silanes to hydrolyze and cross-link to bring the silica beads together to form porous substrates. The curing step can be performed at a much lower temperature (e.g., from room temperature to about 200° C.) than that required for conventional fusion methods (e.g., at about 700° C.). This permits alternative materials (e.g., polymeric materials) to be used as substrate supports for porous coatings. In many examples, a thin layer of TiO₂ or SiO₂ can be first deposited onto a polymeric support to enhance the adhesion of the porous coatings. The polymeric support can also contain channels at predefined locations to provide access to the porous coatings. The use of polymeric materials allows for low-cost manufacturing of flow-through microplates. In addition, the use of cross-linkable silanes that contain desired functional groups (e.g., amines or epoxides) can potentially eliminate the step for surface coating.

Silica beads or particles that are suitable for this purpose include, but are not limited to, silica frits or pure silica. Other porous silica materials, such as those described in WO0061282 and WO0116376, can also be used to prepare silica beads or particles. Solvents suitable for suspending these silica beads include, but are not limited to, texanol/enphos PVB or isopropanol. Other organic solvents may also be used, as appreciated by those of ordinary skill in the art. Examples of silanes that are suitable for this purpose include, but are not limited to, 3-acyloxypropyl-trimethoxysilane, allyltrichlorosilane, 3-aminpropyltriethoxysilane, N-(6-aminohexyl)aminopropyl-trimethoxysilane, bis(triethoxysilye)methane, 2-(3-cyclohexenyl) ethyl)triethoxysilane, 3-glycidoxypropyl-trimethoxysilane, tetramethoxysilane, or a combination thereof. Other silanes that have controllable cross-linking properties and reactivities with silica beads can also be used. In one example, the concentration of the silane(s) employed in the present invention is in the range of from about 0.01% to about 10% by volume. The selection of suitable silanes may depend on particular applications. For example, aminosilane can be used for generating a porous substrate with amine functionality for making DNA or protein microarrays.

A mixture containing both silica beads and silane(s) can be printed or deposited to predefined regions on a substrate support using any conventional means. An example of these methods is based on screen printing technology which uses a silk screen containing domains with a certain mesh size. Many types of substrate supports can be used, such as polymeric supports or inorganic or metal-based supports. Where a polymeric support is used, a layer(s) of SiO₂ or TiO₂ can be deposited prior to the porous coating to enhance the adhesion of the porous material. Channels at defined locations (e.g., underneath the deposited silica-bead patches) can be readily created in the substrate support using methods described above. Substrate supports without flow-through channels may also be utilized in the present invention.

The curing step typical includes a low-temperature (e.g., from room temperature to about 200° C.) treatment to accelerate the cross linking as well as eliminate trace organic byproducts due to the hydrolysis of the silane molecules. A substrate support containing spotted silica beads/silane mixtures can also be stored in a chamber with controlled humidity (e.g., at relative humidity from 30% to 70%) before the curing step to enhance cross-linking.

II. Arrays and Applications Thereof

The porous substrates prepared by the present invention can be used to make arrays. Examples of these arrays include, but are not limited to, nucleic acid arrays, protein arrays, cell arrays, tissue arrays, or membrane arrays. Any array format may be used, such as microarrays, bead arrays, or multi-well microplates. Each array of the present invention comprises a plurality of discrete regions, and each discrete region has a predefined or determinable location on the array. These discrete regions can be organized in various forms or patterns. For instance, the discrete regions can be arranged as an array of regularly spaced areas. Other regular or irregular patterns, such as linear, concentric or spiral patterns, can also be used.

The discrete regions on an array of the present invention may have any size, shape or density. For instance, the shape of a discrete region can be square, ellipsoid, rectangle, triangle, circle, or any other regular or irregular geometric shape, or a portion or combination thereof. For another instance, a discrete region can have a surface area of less than 10, 1, 10⁻¹, 10⁻², 10⁻³, 10⁻⁴, 10⁻⁵, 10⁻⁶, or 10⁻⁷ cm², and the spacing between each discrete region and its closest neighbor, measured from center-to-center, can be in the range of from less than about 10 μm to over about 1 cm. The density of these discrete regions on an array of the present invention may range, without limitation, from less than 10 to over 50,000 regions/cm².

Each discrete region may comprise or be stably associated with a porous substrate for attachment of nucleic acid probes, antibodies, high-affinity binders, cellular components, tissue parts, or other desired biological materials. Any method known in the art may be used to stably attach probes or biological materials to a porous substrate of the present invention. By “stably,” it means that a molecule or cell/tissue component that is attached to a discrete region retains its position relative to the discrete region during array hybridization or reaction.

In many embodiments, an array of the present invention has a flow-through configuration, such that samples or wash buffers can access to the attached porous substrates from both sides of the array. This flow-through design can significantly improve the washability of the porous substrates, and facilitate automation of the washing and drying steps after array hybridization or reaction. In one example, a porous microplate of the present invention (e.g., FIGS. 1A-1C, 2A-2B and 3A-3B) is washed and dried either sequentially or simultaneously after an array-based binding assay. External forces such as vacuum from the bottom side, or pressures applied through the channels in the substrate supports, can be utilized to remove solutions in each porous substrate patch.

In one embodiment, biological membranes or other amphiphilic molecule complexes are attached to the porous substrates of an array of the present invention. Example methods for depositing a membrane onto a substrate surface are described in U.S. Patent Application Publications US20020094544 and US20020019015, and U.S. Provisional Application entitled “Membrane Arrays and Methods of Manufacture” (by Yulong Hong, et al.), all of which are incorporated herein by reference in their entireties. Biological membranes suitable for the present invention include, but are not limited, plasma membranes, nuclear membranes, or cell organelle membranes (e.g., mitochondria or chloroplast membranes). These biological membranes can be isolated from cells or tissues using conventional techniques.

Amphiphilic molecule complexes suitable for the present invention include, but are not limited to, micelle membranes, liposome membranes, amphiphilic molecule bilayers, or vesicle membranes. These membrane structures can be naturally occurring, or assembled in vitro. They can be unilamellar or multilamellar. Other forms of membrane structures can also be used for the present invention.

A membrane structure employed in the present invention can be made from many types of amphiphilic molecules, such as lipids, detergents, surfactants, fatty acid derivatives, or other molecules that have hydrophilic and hydrophobic groups. Specific examples of suitable amphiphilic molecules include, but are not limited to, phosphatidic acid, phosphatidylethanolamine, phosphatidylcholine, phosphatidylinositols, phosphatidylglycerol, sphingomylelin, cardiolipin, lecithin, phosphatidylserine, cephalin, cerebrosides, dicetylphosphate, steroids, terpenes, stearylamine, dodecylamine, hexadecylamine, acetylpalmitate, glycerol ricinoleate, hexadecyl stearate, isopropyl myristate, dioctadecylammonium bromide, amphoteric polymers, triethanolamine lauryl sulfate and cationic lipids, 1-alkyl-2-acyl-phosphoglycerides, and 1-alkyl-1-enyl-2-acyl-phosphoglycerides.

In many instances, the membrane attached to a porous substrate comprises one or more membrane proteins. These membrane proteins can be peripheral or integral membrane proteins. Examples of these membrane proteins include, but are not limited to, receptors (e.g., GPCRs), ion channels, kinases, enzymes, transporters, structural proteins, lipoprotein, glycoproteins, or subunits or fragments thereof. The association between a membrane protein and a membrane can be mediated by a transmembrane sequence (e.g. α-helices comprising multiple hydrophobic amino acids, such as Leu, Ile, Val or Phe), or by a covalently-linked hydrophobic anchor (e.g. a C14 myristic acid via an amide linkage to an amino-terminal Gly, a C16 palmitic acid via thioester to Cys or hydroxyester to Ser or Thr, and a glycosyl phosphatidyl inositol anchor).

A membrane protein can be a naturally-occurring protein in an isolated biological membrane. Attachment of the biological membrane to an array of the present invention also couples the membrane protein to the array. A membrane protein can also be incorporated into a membrane using various techniques. In one embodiment, a membrane protein is premixed with a phospholipid or another amphiphilic molecule before a membrane is created. In another embodiment, a membrane protein is added to a membrane after the membrane is formed. For instance, a membrane may include a lipid containing a streptavidin group. A membrane protein containing a biotin group can be incorporated into the membrane via the specific interaction between biotin and streptavidin. In still another embodiment, a membrane may include a reactive phospholipid, such as phosphatidylenthanolamine. A protein having a complementary reactive group can be incorporated into the membrane by reacting with the phospholipid. For incorporating a protein into a membrane or membrane-like structure, see, for example, Schoch et al., J. RECEPT. RES. 4: 189-200 (1984); Sigel et al., NEUROSCI. LETT. 61: 165-170 (1985); Fujioka et al., BIOCHEM. BIOPHYS, RES. COMM. 156: 54-60 (1988); Lundahl and Yang, J. CHROMATOGR. 544: 283-304 (1991); Dunn et al., BIOCHEMISTRY 28: 2545-2551 (1989); Gomathi and Sharma, FEBS LETT. 330: 146-150 (1993); Gioannini et al., BIOCHEM. BIOPHYS, RES. COMM. 194: 901-908 (1993); and Balen et al., BIOCHEMISTRY 33: 1539-1544 (1994), all of which are incorporated herein by reference.

In one example, a membrane is immobilized to a porous substrate on a flow-through array of the present invention (see, e.g., FIGS. 1A-1C, 2A-2B and 3A-3B). The immobilized membrane includes a membrane protein, such as a GPCR or an ion channel. Functional analyses can be performed to identify modulators of these membrane proteins. A functional assay typically comprises contacting the ligand binding domain of the membrane protein with a candidate molecule, followed by detecting the activation or inactivation of the membrane protein. The candidate molecule may be an agonist, an antagonist, an inhibitor, or an activator of the membrane protein. The activation or inactivation of a membrane protein, such as a GPCR, an ion channel or a membrane-bound kinase, can be detected using conventional techniques, such as by monitoring the activation/inactivation of downstream effectors (e.g., phospholipases, kinases, or phosphatase), the level of second messengers, the change in membrane potentials, or other downstream events. Reagents for detecting ligand binding or assessing the activation/inactivation of membrane proteins can be simultaneously applied to the respective side of the immobilized membrane without interfering with the reactions occurred on the other side of the membrane. This can be achieved, for example, by adding one assay solution to the top side of the immobilized membrane and, at the same time, providing another solution to the bottom side of the membrane through, for example, capillary force or microchannels in the substrate support. Assay reagents for detecting ligand binding or assessing the activation/inactivation of membrane proteins can also be provided to the membrane in the same sample. In certain cases, these reagents may also be provided to the membrane sequentially.

Membranes attached to an array of the present invention can be stabilized by various means. For instance, certain surface chemistries can be employed to enhance the immobilization of biological membranes. Water-soluble proteins can also be utilized to stabilize the membrane microspots. These immobilized membranes can be stored and used not only in an aqueous environment but also in an environment in which the membranes are exposed to air under ambient or controlled humidity.

Several factors can significantly affect the manufacturing and performance of an array, including the size, uniformity and stability of the membrane spots, as well as the functionality and ligand-binding specificity of the associated membrane-proteins. These factors include, for example, printing conditions, printing ink compositions, surface chemistries, bioassay conditions, and receptor quality. Among these factors, surface chemistry plays a major role in determining the quality and bioassay potential of a membrane array. The structures and functions of the lipid molecules and membrane proteins that are immobilized on a surface often depend on the chemical nature of the surface. In addition, for non-flow-through arrays comprising immobilized two-dimensional membranes, the inherent geometric constraints often limit the potential applications of these arrays. For example, for GPCR arrays, an agonist screening typically involves the binding of ligands on one side of the membrane, and the detection of activation/inactivation of the receptor on the other side. The two-dimensional immobilization of GPCRs prevents or makes difficult the simultaneous detection of events on both sides of the membrane.

To address these issues and to improve array performance, the present invention provides arrays that allow for three-dimensional immobilization of membranes on the arrays. This can be achieved by mixing membranes with sol-gel precursors to form membrane-containing sol-gels, followed by depositing these sol-gels into discrete regions on an array surface. Three-dimensional immobilization can also be achieved by using the following three-step process: (1) pre-mixing sol-gel precursors with membranes under conditions that no significant gelation takes place; (2) depositing the mixture of the membranes and sol-gel precursors into discrete regions on an array surface; and (3) treating the array to allow gelation within the discrete regions (such as, by using vapor phase proton-induced gelation approach under controlled humidity). The discrete regions may or may not be connected with flow-through channels, and the sol-gels may or may not be deposited to porous substrates.

Examples of sol-gel precursors suitable for the present invention include, but are not limited to, tetraalkoxysilane, (such as tetraethoxysilane), or trialkoxysilane (such as methyltrimethoxysilane, PEG-silane (2 -(methoxy(polyethyleneoxy)propyl)-trimethoxysilane, or 3-aminopropyltriethoxysilane). The silane monomers with trimethoxy or triethoxy group are stable on the time scale of hours or even days in neutral aqueous solutions. However, at low or high pH, they hydrolyze rapidly (within minutes) to form reactive species that polymerize into silicon gels (sol-gels). Different monomers hydrolyze to a gel with different kinetics. For example, without special precautions tetraethoxysilane hydrolyzes to a gel in about 10 days; tetramethoxysilane in about 2 days; tetra-n-butoxysilane in about 26 days. Acid-catalyzed hydrolysis generally proceeds more rapidly than base hydrolysis, and leads to more linear polymers than base hydrolysis. Therefore, by choosing right sol-gel precursors in combination with proper gelation conditions, one can control gelation kinetics of sol-gel precursors. This allows one to print membrane-containing sol-gels without clogging the pins, thereby creating stronger adhesion between the sol-gels and the surface.

Sol-gel precursors can be hydrolyzed first and then formulated with membranes to form membrane-containing sol-gels before being deposited onto an array surface. This approach is relatively simple. However, due to the gelation kinetics and the size of the sol gels thus-formed, printing of these sol-gels may be difficult in certain cases. Alternatively, sol-gel precursors can be first premixed with membranes under conditions that no significant gelation takes place, followed by depositing the mixture into predefined regions on an array surface, followed by treating the array to initiate gelation. Many methods are available for inducing gelation in the predefined regions. One example is the vapor phase acid or base-induced gelation approach under controlled humidity. In this method, an array that comprises the mixtures of membranes and sol-gel precursors is incubated under high humidity within a container that contains a solution of about 37% hydrochloride acid, or concentrated acetic acid, or aqueous ammonia, or ammonium carbonate. Another example is to treat the array with a basic or acidic solution to allow gelation taking place within the defined regions. Other immobilization, gelation and encapsulation approaches can also be used. See, for example, Gill and Ballesteros, J. AM. CHEM. SOC., 120:8587-8598 (1998), and Arkles, Silanes, SILICONES AND METAL-ORGANICS, Gelest Catalog (1998), both of which are incorporated herein by reference in their entireties.

The above-described three-dimensional immobilization approach allows attachment of a high load of membranes within predefined regions on an array surface. This provides significant advantages over many other membrane arrays. For instance, the three-dimensional approach can offer improved detection sensitivity or specificity, and better suitability for functional assays of membrane proteins. Moreover, the use of mixtures of membranes and sol-gel precursors can stabilize the attachment of membranes to an array surface due to silanization reaction with the surface (such as a bare glass surface or a silane-modified surface, e.g., a GAPS or epoxy-silane surface). This can not only increase the mechanical stability of the arrays, but also eliminate the requirement for stable surface chemistry for immobilization of membranes.

In addition to membranes or membrane-like structures, nucleic acid or polypeptide probes can also be immobilized to discrete regions on an array of the present invention. In many instances, these discrete regions comprise or are coated with porous substrates, or connected with flow-through channels. Nucleic acid probes suitable for the present invention include, but are not limited to, DNA, RNA, PNA (“Peptide Nucleic Acid”), or modified forms thereof. The nucleotide units in each nucleic acid probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the nucleic acid probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

In many cases, perfect mismatch probes are also included for each perfect match probe on an array of the present invention. A perfect mismatch probe has the same sequence as the corresponding perfect match probe except for a homomeric substitution (i.e., A to T, T to A, G to C, or C to G) at or near the center of the perfect mismatch probe. For instance, if the perfect match probe has 2n nucleotide residues, the homomeric substitution in the corresponding perfect mismatch probe is either at the n or n+1 position, but not at both positions. Where the perfect match probe has 2n+1 nucleotide residues, the homomeric substitution in the corresponding perfect mismatch probe is at the n+1 position.

Any conventional method can be used to spot or deposit nucleic acid probes on a porous substrate. For instance, the probes can be synthesized in a step-by-step manner on a porous substrate, or can be attached to the porous substrate in pre-synthesized forms. Algorithms for reducing the number of synthesis cycles can be used. In one embodiment, an array of the present invention is synthesized in a combinational fashion by delivering nucleotide monomers to the discrete regions on the array through mechanically constrained flowpaths. In another embodiment, an array of the present invention is synthesized by spotting nucleotide monomer reagents onto the porous substrates on the array using an ink jet printer. In yet another embodiment, polynucleotide probes are immobilized to an array by using photolithography techniques.

Antibodies or antibody-like molecules can also be spotted or deposited to the porous substrates on an array of the present invention. Suitable antibodies include, for example, polyclonal antibodies, monoclonal antibodies, chimeric antibodies, single chain antibodies, synthetic antibodies, Fab fragments, or fragments produced by a Fab expression library. Other peptides, scaffolds, antibody mimics, high-affinity binders, or protein-binding ligands can also be used to construct the arrays of the present invention.

Numerous methods are available for immobilizing antibodies or other polypeptide probes on a substrate. Examples of these methods include, but are not limited to, diffusion (e.g., agarose or polyacrylamide gel), surface absorption (e.g., nitrocellulose or PVDF), covalent binding (e.g., silanes or aldehyde), or non-covalent affinity binding (e.g., biotin-streptavidin). Examples of protein array fabrication methods include, but are not limited to, ink-jetting, robotic contact printing, photolithography, or piezoelectric spotting. The method described in MacBeath and Schreiber, SCIENCE, 289: 1760-1763 (2000) can also be used.

Probes or other agents used in an array assay can be conjugated, either covalently or non-covalently, with one or more labeling moieties. These labeling moieties can include compositions that are detectable by optical, spectroscopic, photochemical, biochemical, bioelectronic, immunochemical, electrical, chemical or other means. Examples of suitable labeling moieties include radioisotopes, chemiluminescent compounds, labeled binding ligands, labeled agonists or antagonists, heavy metal atoms, spectroscopic markers, such as fluorescent markers or dyes, magnetic labels, linked enzymes, mass spectrometry tags, spin labels, electron transfer donors and acceptors, and the like.

Array analyses can be performed in absolute or differential formats. In an absolute format, each single reading collects signals for only one label, and in a differential format, at least two different labels can be read at the same time. Two commonly used fluorescent labels for differential formats are Cy3 and Cy5. These are fluorophores that, once excited with optical light of 500-700 nm wavelength (550 and 649 nm, respectively), emit photons of a lower wavelength shifted by about 20 nm. An optical wavelength filter tuned to the emission wavelength allows rejection of any stray excitation light and the selective detection of the fluorescent signal.

More recently, europium-chelate labels have been developed to allow for a larger shift between the excitation wavelength of 351 nm and the detection wavelength of 615 nm. This increased Stokes shift allows for easier discrimination between the excitation and fluorescence wavelengths. However, the 351 nm excitation wavelength needed is not compatible with standard biological-assay reader-instrumentation and substrate materials, since this UV wavelength causes auto-fluorescence in most commonly used glasses. Even small impurity levels can cause an autofluorescence signal which increases the background during the measurement.

The present invention features the use of UV-compatible porous substrate materials (such as those made from monodispersed silica spheres) for biological assays that rely on detection of fluorescently labeled molecules such as cDNA, proteins, or lipids. Examples of UV-compatible materials include, but are not limited to, silica-based glass, fused silica, calcium fluoride or sapphire. This UV compatibility allows for reduced background signal and consequently enhanced detection sensitivity.

As described above, enhanced signal can be obtained when array experiments are performed on a porous substrate material versus a flat glass surface. This signal enhancement is partially attributed to the increased effective assay volume as well as to the increased scattering facilitated by the micrometer-scale surface particles. The increased scattering increases the effective optical path length of a photon impinging on the substrate device. The increased photon pathlength causes an increase in the probability that the photon encounters an optically active atom absorbing at the photon's wavelength. This effect leads to a larger absorption probability of the optically active label and hence to increased emission of down-converted fluorescence photons. While the above analysis is of general validity, other material properties, such as intrinsic material absorption, play an important role in the achieved signal to noise. When the excitation wavelengths are in the ultra-violet wavelength range, certain materials auto-fluoresce. The use of UV-compatible porous substrates can significantly reduce background fluorescence when illuminated with UV light. This leads to better signal to noise and enhanced signal sensitivity. See Example 2.

In addition to the use of traditional substrate supports, such as 1737 glass (Corning Inc.), the present invention also contemplates the use of UV-compatible substrate supports for holding or immobilizing UV-compatible porous materials. These UV-compatible substrate supports can be prepared using the same materials that are employed for making the UV-compatible porous substrates.

Signals gathered from an array of the present invention can be analyzed using commercially available or in-house designed software. Controls, such as for scan sensitivity, probe labeling and sample quantitation, can be included in the same or parallel experiments. Signals can be scaled or normalized before being subject to further analysis.

It should be understood that the above-described embodiments and the following examples are given by way of illustration, not limitation. Various changes and modifications within the scope of the present invention will become apparent to those skilled in the art from the present description.

III. Examples

### EXAMPLE 1

### GPCR Assays Using Flow-Through Microplates With Porous Substrates

96 patches of silica-based porous substrate (GAPS™, Corning Inc.) were screen printed onto a 1737 glass support plate, and sintered at 695° C. The glass support was pre-fabricated by sand blasting to generate 192 microchannels. Under each porous patch, there are two microchannels which provide access to the porous patch from the opposite side of the glass support. Human muscarinic receptor subtype 1 (M1), delta opioid receptor subtype 2 (delta2), and muscarinic receptor subtype 2 (M2) were printed onto the GAPS™-porous substrate with a configuration such that each receptor was aligned in one column with four replicates (i.e., columns 1, 2 and 3 for M1, delta2 and M2 receptors, respectively; see FIG. 4A). This array was then treated with a cocktail of labeled ligands containing 2 nM Cy3B-telenzepine and 4 nM Cy5-naltrexone in the absence (FIG. 4A) or presence of (FIG. 4B) unlabeled telenzepine (2 μM) and naltrexone (4 μM). Telenzepine is an antagonist of M1 and M2 receptors, and naltrexone is an antagonist of delta2 receptor. Telenzepine and naltrexone can bind to M1/M2 and delta2 receptors, respectively. After one hour incubation, a vacuum force was applied to remove the assay solution and the sequential washing solution before the array was finally being dried.

FIG. 4A indicates strong binding between Cy3B-telenzepine and M1 receptor (column 1, green) and between Cy5-naltrexone and delta2 receptor (column 2, red). Weak binding signals were observed between Cy3B-telenzepine and M2 receptor (column 3, green). These bindings were inhibited by unlabeled telenzepine and naltrexone, suggesting specific interactions between the ligands and their respective receptors.

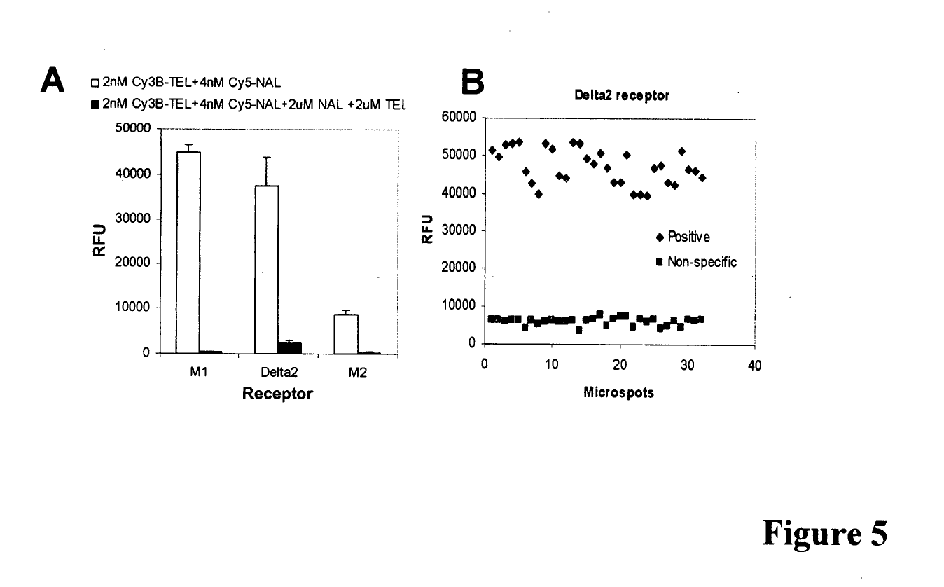

FIGS. 5A and 5B further demonstrate the superior performance of flow-through microplates for GPCR arrays on GAPS porous substrates. FIG. 5A is a diagram showing the average fluorescence intensities of three different receptors (M1, delta2 and M2) after assayed with the cocktail ligands in the absence or presence of unlabeled compounds (see FIGS. 4A and 4B). FIG. 5B indicates the fluorescence intensities of delta2 receptor in the array assays of FIGS. 4A and 4B as a function of microspots treated with the cocktail ligands in the absence (referred to “Positive”) and presence (“non-specific”) of unlabeled compounds. The total binding signals of receptor microspots on the GAPS-porous substrate after binding assays are 20 times stronger than those of corresponding receptor microspots fabricated on 2-D GAPSII slide (Corning Inc) under the same assay and image acquisition conditions, suggesting a higher loading capacity of porous substrates than the 2-D surfaces. In addition, the array performance, measured by the assay variation (CV) and binding specificity, of GPCR arrays on flow-through microplate with porous substrates are significantly better than that on porous substrates without flow through channels using the same assay protocol except for the washing step (it is automatically vacuum washing/drying step for the flow-through microplate with porous substrate, instead of conventional solution washing followed by blown drying for arrays on porous substrates). The CV for M1 receptors in microarrays fabricated on porous substrate with follow through configuration was less than 10%, compared to about 20% on conventional porous substrates.

### EXAMPLE 2

### UV-Compatible Porous Substrates

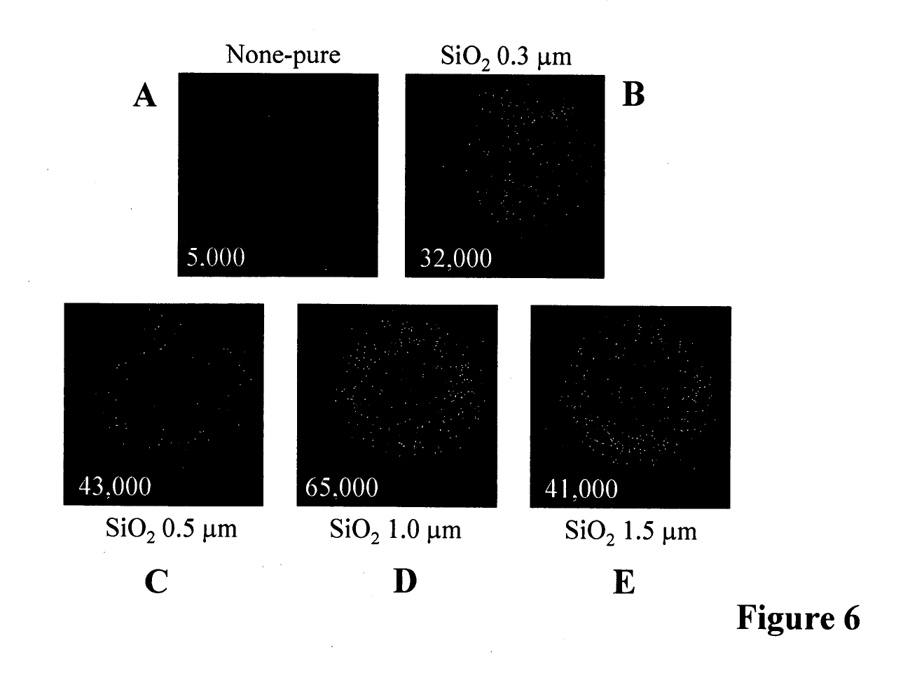

Experiments with various substrate materials showed that significantly lower background signal is achieved when UV-compatible substrates were used. The reduction in background signal can be as much as a factor of 10 or more between pristine fused silica and pristine conventional 1737 glass substrates. This dramatic improvement in background suppression can readily be leveraged if the new substrate can be made into a porous layer to allow for increased signal levels due to increased excitation scatter and volume. To demonstrate feasibility, 2 mg of mono-dispersed fused silica powder was mixed with 1 micro-liter of diluted europium chelate solution. The mixture was then hand-spotted onto a fused silica substrate support. Using a fluorescence imaging setup, the fluorescence signal from each spot was analyzed under conditions of equal excitation intensity. It was observed that mixing the europium chelates with silica powder increases the signal by 800-1300% (FIGS. 6A-6E). Furthermore, the largest fluorescence enhancement was detected when the europium chelates were mixed with 1.0-μm particle size fused-silica (compare FIG. 6D with FIGS. 6A-6C and 6E).

A subsequent background measurement of a fused-silica coated substrate support showed that the fused silica powder does not increase the background noise when compared to bulk silica. This suggests that a porous fused silica coating can yield increased europium fluorescence while maintaining a low background noise.

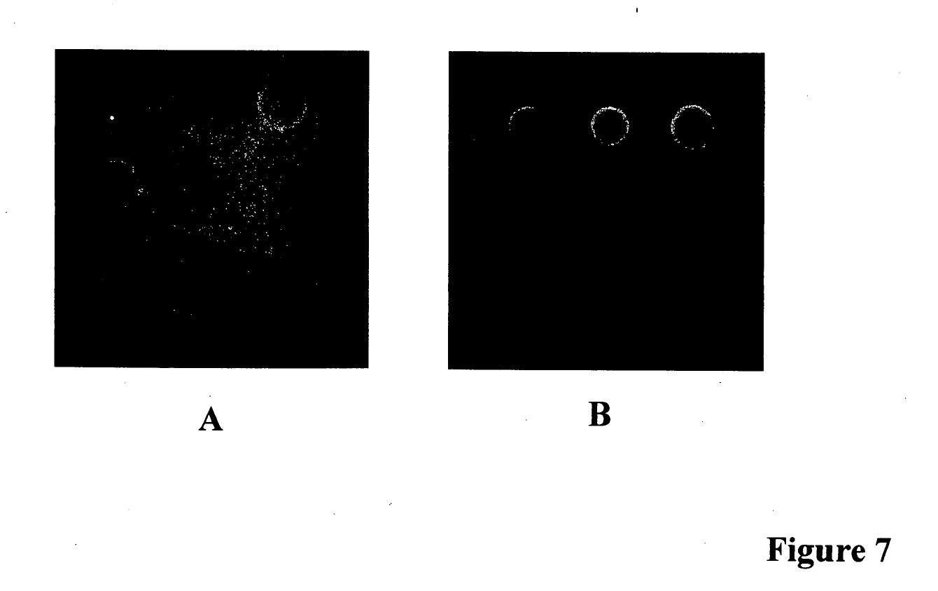

In order to confirm these results for true porous substrates, screen-printed versions of both the traditional (1737) and fused silica compositions were made, using nominally about 11m sized particles. These samples were then printed with several spots of 1:1000 diluted eu-GTP dye (Perkin Elmer) using a Cartesian printer with a CMP-3 quill pin and a minimal dwell time (20 ms). The samples were again imaged using a time-resolved fluorescence instrument, and the results are shown in FIGS. 7A and 7B. While the maximum intensity is similar for both samples, the background of the fused silica sample (FIG. 7B) is nearly half that of the traditional material (FIG. 7A). When the average signal-to-noise (S/N) is calculated for the three spots common to both samples, the traditional sample registers a S/N=1.079, while the fused silica sample S/N=1.557.

### EXAMPLE 3

### Sample Processing of UV-Compatible Porous Substrates

Experimental work on cDNA microarrays showed that porous glass coatings with thickness in the range 10-40 μm and pore/particle size from 0.8-1.2 μm were nearly optimal among many tested samples. Given the data of FIGS. 6A-6E, this also seems to be an optimal range for fused silica used with eu-GTP dye. Therefore, the objective of this Example is to fabricate a similar fused silica coating that is mechanically robust.

Tape-casting was selected as a method for preparing the coating. Slip for tape casting was prepared by first dispersing 30 g of optical grade, monodisperse silica spheres measuring 1 μm in diameter and manufactured by GelTech in 30 g of isopropanol on a vibratory mill. No dispersants or surfactants were used in making the slip to avoid unnecessary contamination of the fused silica that might lead to either unwanted fluorescence or devitrification on firing. After 24 hours of vibratory mixing, the slurry was transferred to a glass bottle, and 2 g of Butvar B-98 polyvinylbutyral from Solutia to act as a binder were added. The mixture was homogenized for 72 hours prior to use.

Billets of fused silica that measure 0.25×1×2.5 inches were tiled onto a small (10×12 in) and held in place by “double-stick” tape. Tape-casting slip was coating onto the billets using a blade with a 4 mil gap height. The coating was allowed to dry in place. The billet with the most uniform looking coating was fired at 1150° C. for 30 minutes. Thickness of the coating is estimated to be approximately 25 μm.

While initial samples were tape-cast, FIGS. 7A and 7B indicate that high-quality screen-printed samples have also been produced, and these can result in even more repeatable surface quality. Porous fused silica coatings of 1 μm silica spheres (GelTech Inc) were applied to as-ground fused silica slides (Corning HPFS) by screen printing according to the following procedure. Screen printing ink was prepared by first dissolving 1.5 w/o (water-in-oil) polyvinyl butyral (Butvar B-98, Solutia Inc.) in Texanol (2,2,4-trimethyl-1,3-pentanediolmono-(2-methylpropanoate), Acros Organics). The solution/vehicle was stirred over medium heat, 50° C., for 48 hours to thoroughly homogenize before use. A weight of silica spheres that gives a 50 volume percent mixture was added to the vehicle. The mixture was stirred initially with a plastic spatula. Final mixing and addition of more vehicle was performed on a three-roll-mill. Addition of more vehicle was to achieve rheological characteristics consistent with a screen printing ink. Viscosity of the ink was measured to be about 20,000 cps on a Brookfield viscometer.

Screens used for printing were made by IRI/Alpha Metal of Johnson City, N.Y. and consisted of a 5×5 inch square frame, 230 mesh stainless steel wire mesh with a 1.4 mil wire diameter, and a 75 μm emulsion. Screen printing was performed on a custom-made printer onto as-ground fused silica slides. Snap height, distance between the top surface of the slide to be coated and the bottom of the screen, was measured to be about 55 mil. A squeegee pressure of 20 lb was selected, but squeegee speed could not be independently controlled. Slides were placed horizontally in a drying oven immediately after printing and dried overnight at 75° C. Coated slides were fired according to the following schedule: linear ramp to 1100° C. in 3.6 hours, hold at 1100° C. for 30 minutes, cool to room temperature in 3.6 hours (likely longer due to thermal mass of furnace). Following firing, slides were placed in a vacuum dessicator prior to GAPS coating.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

What is claimed is:

1. An array comprising at least one substrate support and a plurality of discrete regions, each said discrete region comprising a porous substrate attached to or supported by said at least one substrate support, and said porous substrate being accessible from at least two sides of said array.

2. The array of claim 1, wherein said porous substrate is attached to or supported by a surface of said substrate support, and wherein said substrate support comprises at least one channel which passes through said substrate support from said surface to a surface opposite thereto, and a wash buffer is capable of communicating from said porous substrate to said opposite surface through said channel.

3. The array of claim 1, wherein said substrate support comprises a multi-well microplate, and said porous substrate resides in a well of said microplate, and wherein said substrate support comprises at least one channel which passes through said substrate support from a bottom surface thereof to said well, and a wash buffer is capable of communicating from said bottom surface to said porous substrate through said channel.

4. The array of claim 1, wherein said substrate support comprises a holey microplate including a plurality of openings, and said porous substrate is positioned in one of said openings.

5. The array of claim 1, wherein said at least one substrate support comprises two holey plates, and said porous substrate is part of a porous material sheet which is sandwiched between said two holey plates, and wherein said two holey plates are aligned such that a wash buffer is capable of accessing to said porous substrate from both sides of said array.

6. The array of claim 1, wherein said porous substrate comprises anodic aluminum oxide, fused silica or sol-gel.

7. The array of claim 6, wherein said porous substrate is stably associated with a membrane, a nucleic acid, a polypeptide, a polysaccharide, a lipid, a cell, a cell component, a tissue, or a tissue part.

8. The array of claim 6, wherein said porous substrate is stably associated with a membrane comprising a transmembrane protein.

9. The array of claim 6, wherein said porous substrate is stably associated with a membrane comprising a membrane protein selected from the group consisting of a G protein coupled receptor, an ion channel, a transporter, and a kinase receptor.

10. The array of claim 1, wherein said porous substrate is a gelation product of a mixture comprising a sol-gel precursor and a membrane.

11. The array of claim 10, wherein said sol-gel precursor is a tetraalkoxysilane or a trialkoxysilane.

12. The array of claim 1, wherein said porous substrate is a fusion product of a mixture comprising silica beads and at least one silane.

13. The array of claim 12, wherein said silane is selected from the group consisting of 3-acyloxypropyl-trimethoxysilane, allyltrichlorosilane, 3-aminpropyltriethoxysilane, N-(6-aminohexyl)aminopropyl-trimethoxysilane, bis(triethoxysilye)methane, 2-(3-cyclohexenyl)ethyl) triethoxysilane, 3-glycidoxypropyl-trimethoxysilane, and tetramethoxysilane.

14. The array of claim 1, wherein said porous substrate is UV-compatible.

15. The array of claim 14, wherein said porous substrate comprises fused silica, calcium fluoride or sapphire.

16. The array of claim 1, wherein said porous substrate consists essentially of substantially pure fused silica.

17. An array comprising a substrate support including a plurality of discrete regions, each said discrete region comprising a porous substrate which is a fusion product of a mixture comprising silica beads and at least one silane.

18. The array of claim 17, wherein said porous substrate is prepared by a method comprising the steps:
formulating said silica beads in an organic solvent comprising said at least one silane;
depositing said formulated silica beads in one of said discrete regions; and
fusing said silica beads to form said porous substrate.

19. The array of claim 18, wherein said fusing is performed at a temperature of no greater than about 200° C.

20. The array of claim 18, wherein said silane is selected from the group consisting of 3-acyloxypropyl-trimethoxysilane, allyltrichlorosilane, 3-aminpropyltriethoxysilane, N-(6-aminohexyl)aminopropyl-trimethoxysilane, bis(triethoxysilye)methane, 2-(3-cyclohexenyl)ethyl) triethoxysilane, 3-glycidoxypropyl-trimethoxysilane, and tetramethoxysilane.

21. The array of claim 18, wherein the concentration of said at least one silane in said mixture is from about 0.01% to about 10% by volume.

22. The array of claim 18, wherein said substrate support comprises a polymeric material, an inorganic material, or a metal.

23. The array of claim 18, wherein said porous substrate is stably associated with a surface of said substrate support, and said substrate support comprises at least one channel which passes through said substrate support from said surface thereof to a surface opposite thereto, and wherein a wash buffer is capable of communicating from said opposite surface to said porous substrate through said channel.

24. The array of claim 18, wherein said porous substrate is stably associated with a membrane, a nucleic acid, a polypeptide, a polysaccharide, a lipid, a cell, a cell component, a tissue, or a tissue part.

25. A method of fabricating an array, comprising the steps of:
formulating silica beads in an organic solvent comprising said at least one silane;
depositing said formulated silica beads in discrete regions of a substrate support; and
fusing said silica beads to form porous substrates in said discrete regions.

26. An array comprising a substrate support including a plurality of discrete regions, each of which comprises a gelation product of a mixture comprising at least one sol-gel precursor and a membrane.

27. The array of claim 26, wherein said gelation product is prepared by a method comprising the steps of:
mixing said at least one sol-gel precursor with said membrane;
hydrolyzing said at least one sol-gel precursor to form a sol-gel including said membrane; and
depositing said sol-gel into said discrete regions.

28. The array of claim 26, wherein said gelation product is prepared by a method comprising the steps of:
mixing said at least one sol-gel precursor with said membrane under conditions that no significant gelation occurs;
depositing said mixed sol-gel precursor and membrane into said discrete regions; and
initiating gelation in said discrete regions to form a sol-gel including said membrane.

29. The array according to claim 26, wherein said sol-gel precursor is a tetraalkoxysilane or a trialkoxysilane.

30. The array according to claim 26, wherein said membrane comprises a membrane protein selected from the group consisting of a G protein coupled receptor, an ion channel, a transporter, and a kinase receptor.

31. A method for fabricating an array, comprising the steps of:
mixing at least one sol-gel precursor with a membrane under conditions that no significant gelation occurs;
depositing said mixed sol-gel precursor and membrane into discrete regions on a substrate support; and
initiating gelation in each of said discrete regions to form a sol-gel including said membrane.

32. A method for fabricating an array, comprising the steps of:
mixing at least one sol-gel precursor with a membrane;
hydrolyzing said at least one sol-gel precursor to form a sol-gel including said membrane; and
deposit said sol-gel into discrete regions of a substrate support.

33. An array comprising a substrate support including a plurality of discrete regions, each of which comprises a UV-compatible porous substrate.

34. The array of claim 33, wherein said UV-compatible porous substrate comprises silica-based glass, calcium fluoride or sapphire.

35. The array of claim 33, wherein said UV-compatible porous substrate comprises fused silica.

36. The array of claim 33, wherein said UV-compatible porous substrate consists essentially of substantially pure fused silica.

37. The array of claim 36, wherein said fused silica consists essentially of silica beads with particle sizes of from about 1 nm to about 5 μm.

38. The array of claim 36, wherein said fused silica consists essentially of silica beads with particle sizes of from about 0.3 μm to about 1.5 μm.

39. The array of claim 36, wherein said fused silica consists essentially of silica beads with particle sizes of about 1 μm.

40. The array of claim 33, wherein said substrate support is UV-compatible.

41. The array of claim 40, wherein said substrate support comprises silica-based glass, calcium fluoride or sapphire.

42. The array of claim 40, wherein said substrate support comprises fused silica.

43. The array of claim 40, wherein said substrate support consists essentially of substantially pure fused silica.

44. The array of claim 40, wherein said fused silica consists essentially of silica beads with particle sizes of from about 1 nm to about 5 μm.

45. The array of claim 40, wherein said fused silica consists essentially of silica beads with particle sizes of from about 0.3 μm to about 1.5 μm.

46. The array of claim 40, wherein said fused silica consists essentially of silica beads with particle sizes of about 1 μm.

47. A silica-based porous flow-through microplate fabricated according to the following steps:
producing a plurality of channels by sand blasting or laser drilling at predetermined locations on a glass plate;
depositing patches of silicate frits to said predetermined locations;
sintering to consolidate said frits to form porous substrates; and
assembling the glass plate into a microplate.

48. A flow-through microplate fabricated according to the following steps:
producing a plurality of channels by injection molding at predetermined regions on a substrate support;
reformulating silicate frits with silanes;
depositing patches of sol-gels containing said silicate frits and silanes to the predetermined regions on the substrate support;
consolidating said silicate frits and silanes to form porous substrates; and
assembling the substrate support to form a microplate.

49. A stand-alone porous disc-based microplate fabricated according to the following steps:
injection molding to make a holey microplate, said holey microplate comprising recess areas in predetermined regions on a side wall of a well of said holey microplate;
depositing patches of silicate frits to a substrate support;
consolidating the silicate frits to form standalone porous substrates; and
positioning the standalone porous substrates into the recess areas of the holey microplate.

50. A flow-through polymeric microplate comprising polymeric porous substrates, said polymeric microplate being fabricated according to the following steps:
producing channels by injection molding at predetermined locations on a polymeric substrate support;
positioning polymeric porous substrates to the predetermined locations; and
assembling the polymeric substrate support and the polymeric porous substrates to form a microplate by thermal bonding or adhesive chemistry.

51. A flow-through microplate prepared according to the following steps:
positional etching of a glass substrate to form separate porous substrate patches at predetermined locations such that only a top layer of the glass substrate becomes porous;
sand blasting or laser drilling to prepare at least one channel underneath each said porous substrate patch at the predetermined locations such that the channel passes through the substrate; and
assembling the substrate to form a microplate.
